(19) United States
(12) Patent Application Publication
(10) Pub. No.: US 2023/0135281 A1
(43) Pub. Date: May 4, 2023

(54) CONTROLLED RELEASE OF A THERAPEUTIC FROM AN OPHTHALMIC DEVICE WITH A LOCALLY ENHANCED CONCENTRATION OF CHLORIDE IONS

(71) Applicant: TWENTY TWENTY THERAPEUTICS LLC, South San Francisco, CA (US)

(72) Inventors: Zidong Li, South San Francisco, CA (US); Brian Kim, South San Francisco, CA (US); Christian Gutierrez, Pacifica, CA (US)

(21) Appl. No.: 17/971,871

(22) Filed: Oct. 24, 2022

Related U.S. Application Data
(60) Provisional application No. 63/272,732, filed on Oct. 28, 2021.

(51) Int. Cl. A61N 1/04

(52) U.S. Cl. CPC A61N 1/0448; A61N 1/042

(57) ABSTRACT

An ophthalmic device including a hydrogel-based material body that can encapsulate a reservoir housing a therapeutic and a metal electrode covering the reservoir. The therapeutic can be delivered into an eye by way of electrodissolution of the metal electrode. The electrodissolution can be enhanced by the presence of chloride ions proximal to the metal electrode, and the ophthalmic device can be engineered to ensure the presence of chloride ions proximal to the metal electrode.

### RELATED APPLICATIONS

This application claims priority to U.S. Provisional Application Ser. No. 63/272,732, filed Oct. 28, 2021, entitled “CONTROLLED RELEASE OF A THERAPEUTIC FROM AN OPHTHALMIC DEVICE WITH A LOCALLY ENHANCED CONCENTRATION OF CHLORIDE IONS”. The entirety of this provisional application is hereby incorporated by reference for all purposes.

### TECHNICAL FIELD

The present disclosure relates generally to the controlled release of a therapeutic from an ophthalmic device and, more specifically, to achieving a more predictable controlled release of the therapeutic from the ophthalmic device by locally enhancing the concentration of chloride ions.

### BACKGROUND

Diseases and disorders of a patient's eye can prove difficult to treat with therapeutics delivered by traditional at home delivery methods, such as eye drops, due to problems with precise positioning, dosing, and timing. Ophthalmic devices, such as contact lenses placed directly over the eye, can be used to release therapeutics into the eye in specific quantities and at specific target positions, but timing remains an issue. Generally, such ophthalmic devices can release the therapeutic from confinement using an electrodissolution process, but this electrodissolution process shows limited effectiveness because the materials used in these ophthalmic devices tend to limit saline access during the electrodissolution. The presence of chloride ions from the saline is important for timely and effective electrodissolution.

### SUMMARY

The present disclosure relates to locally enhancing the concentration of chloride ions to control the release of a therapeutic from an ophthalmic device via an electrodissolution process.

In an aspect, the present disclosure includes an ophthalmic device that can deliver a therapeutic to an eye of a subject wearing the device where electrodissolution is enhanced by chloride ions. The ophthalmic device includes a reservoir having an interior configured to hold a therapeutic and a metal electrode configured to cover an opening of the reservoir and to receive an electronic signal that electrodissolves the metal electrode to release the therapeutic from the reservoir. The ophthalmic device also includes a body comprising a silicone-hydrogel or hydrogel-based material that is configured to encapsulate the reservoir and the electrode.

In another aspect, the present disclosure includes a method for releasing a therapeutic to an eye where electrodissolution is enhanced by chloride ions. An ophthalmic device is positioned on an eye of a subject. The ophthalmic device includes a reservoir having an interior configured to hold a therapeutic, a metal electrode configured to cover an opening of the reservoir and to electrodissolve to release the therapeutic from the reservoir; and a body comprising a hydrogel-based material configured to encapsulate the reservoir and the electrode. An electrical signal is applied to the electrode so that the electrode undergoes electrodissolution to release the therapeutic.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 shows a diagram of a system that can deliver a therapeutic to a subject's eye;

FIG. 2 shows an example representation of electrodissolution of a metal electrode that is part of the ophthalmic device of FIG. 1;

FIG. 3 shows examples of the effect of chloride ions on electrodissolution of the metal electrode of the ophthalmic device of FIG. 1;

FIG. 4 shows an example of the ophthalmic device of FIG. 1 in contact with a saline solution;

FIG. 5 shows an example of the ophthalmic device of FIG. 1 including a boost layer;

FIG. 6 shows example reactions of different boost layer configurations in the presence of water;

FIG. 7 shows an example of the ophthalmic device having both an increased permeability to chloride and a boost layer and is in contact with a saline solution;

FIG. 8 is a process flow diagram illustrating a method for controlling the release of a therapeutic from an ophthalmic device in the presence of chloride ions;

FIG. 9 is a process flow diagram illustrating a method for enhancing a local chloride environment in an ophthalmic device; and

FIG. 10 is a process flow diagram illustrating a method for controlling the release of a therapeutic from an ophthalmic device having a locally enhanced chloride environment to treat a subject.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “ophthalmic device” refers to a medical instrument used on or within a portion of a patient's eye for optometry or ophthalmology purposes (e.g., for diagnosis, surgery, vision correction, or the like). An ophthalmic device can be “smart” when it includes one or more components that facilitate one or more active processes for purposes other than traditional lens-based vision correction (e.g., therapeutic release). Unless otherwise stated, as used herein, the term “ophthalmic device” should be understood to mean “smart ophthalmic device”.

As used herein, the term “reservoir” refers to a storehouse for a therapeutic with a portion being open for release of the therapeutic (allowing for diffusion of the therapeutic out of the reservoir and into the surrounding hydrogel matrix). The opening may be covered to prevent release of the therapeutic. In some instances, the covering can facilitate release of the therapeutic from the reservoir. For example, at least a portion of the covering can be an electrode that can electrodissolve to facilitate the release of the therapeutic.

As used herein, the term “therapeutic” refers to one or more substance (e.g., liquid, solid, or gas) related to the treatment, symptom relief, or palliative care of a disease, injury, or other malady. The therapeutic can be a pharmaceutical, for example.

As used herein, the term “electrode” refers to a conductive solid (e.g., including one or more metals, one or more polymers, or the like) that receives/transmits an electrical signal. Unless otherwise noted, the term “metal electrode” is used to refer to the “working electrode” of an electrochemical system, which includes the working electrode and a counter electrode and a reference electrode or a counter/reference electrode. A non-limiting example of an electrode is a thin-film gold electrode.

As used herein, the term “electrical signal” refers to a signal waveform generated by an electronic means, such as a generator. An electrical signal may be a voltage signal or a current signal.

As used herein, the term “electrodissolution” refers to a process for dissolving a solute using an electrical catalyst. In one non-limiting example, application of an electrical signal to a solid metal can cause the solid metal to dissolve into separate molecules.

As used herein, the term “hydrogel-based material” refers to a soft contact lens material, such as a hydrogel or a silicone-hydrogel material, including, but not limited to, all hydrogel and silicone-hydrogel materials. Other materials that may be used in a soft contact lens are also included as or in a hydrogel-based material

As used herein, the term “hydrogel” refers to a crosslinked hydrophilic polymer that does not dissolve in water. A hydrogel is generally highly absorbent yet maintains a well-defined structure.

As used herein, the term “permeability” refers to a characteristic of a material that allows one or more substances to penetrate or pass through the material.

As used herein, the term “boost layer” refers to a layer proximal to or on top of an electrode that can dissolve to create a local environment near at least a portion of the electrode that boosts the speed of the electrodissolution processes.

As used herein, the term “gap layer” refers to a layer between a boost layer and an electrode that can store a substance (liquid, solid, or gas). For example, the gap layer can store a saline solution or create an air pocket.

As used herein, the term “solubility” refers to the ability to be dissolved in the presence of a solvent (liquid, solid, or gas). For example, a water-soluble material has the ability to dissolve in the presence of water.

As used herein, the term “solid salt” refers to any solid-phase chemical compound formed from the reaction of an acid with a base with all or part of the hydrogen of the acid replaced by a metal or other cation.

As used herein, the terms “subject” and “patient” can be used interchangeably and refer to any warm-blooded organism including, but not limited to, a human being, a pig, a rat, a mouse, a dog, a cat, a goat, a sheep, a horse, a monkey, an ape, a rabbit, a cow, etc.

### II. Overview

The present disclosure relates generally to controlling release of a therapeutic from an ophthalmic device. The therapeutic can be held in a reservoir of the ophthalmic device that is covered by a metal electrode (e.g., made of gold or copper). The therapeutic can be released from the ophthalmic device by dissolving the metal electrode into a solute using an electrical signal as a catalyst to drive the reaction using an electrodissolution process. To achieve efficient electrodissolution of the metal electrode, it is necessary to have a quantity of chloride ions, or another halide, proximal the metal electrode at the time the electrical signal is applied. The chloride ions can enhance the electrodissolution process (e.g., without chloride ions, the process may be highly inefficient or ineffective, requiring several minutes to hours, but with chloride ions, the process may take only seconds). However, when the metal electrode is encased within a silicone-hydrogel or hydrogel-based material, chloride ions from a surrounding solution (e.g., tears) are impeded from reaching the surface of the metal electrode rapidly. When a metal electrode embedded within a traditional hydrogel-based material body is electrodissolved an insufficient quantity of chloride ions may have permeated the body, which can result in electrodissolution requiring several minutes to hours. In contrast, when a metal electrode is positioned within a solution comprising chloride ions and electrodissolved, then the electrodissolution of the metal electrode may take only seconds.

As described herein, three techniques can be used to enhance the chloride ion concentration at the local environment of a metal electrode surrounded by a hydrogel-based material within the ophthalmic device. First, a hydrogel-based material with a high enough chloride permeability that a sufficient quantity of chloride ions can permeate the ophthalmic device can be used to support efficient electrodissolution of the metal electrode. Second, a boost layer, made of a solid salt, which includes chloride ions, can be included proximal the metal electrode. The solid salt dissolves when a quantity of water molecules diffuse through the ophthalmic device to create an enhanced chloride ion environment proximal the metal electrode. Third, a boost layer that includes a water-soluble material can be positioned above the metal electrode to form a gap layer. The gap layer can be empty, to create a space for chloride ions within the ophthalmic device to gravitate towards, or can store a chloride containing material, which can be released to create an enhanced chloride ion environment. The boost layer can again dissolve in the presence of a sufficient quantity of water. The first, second, and/or third methods can be used either alone or in combination to enhance the electrodissolution process.

### III. Systems

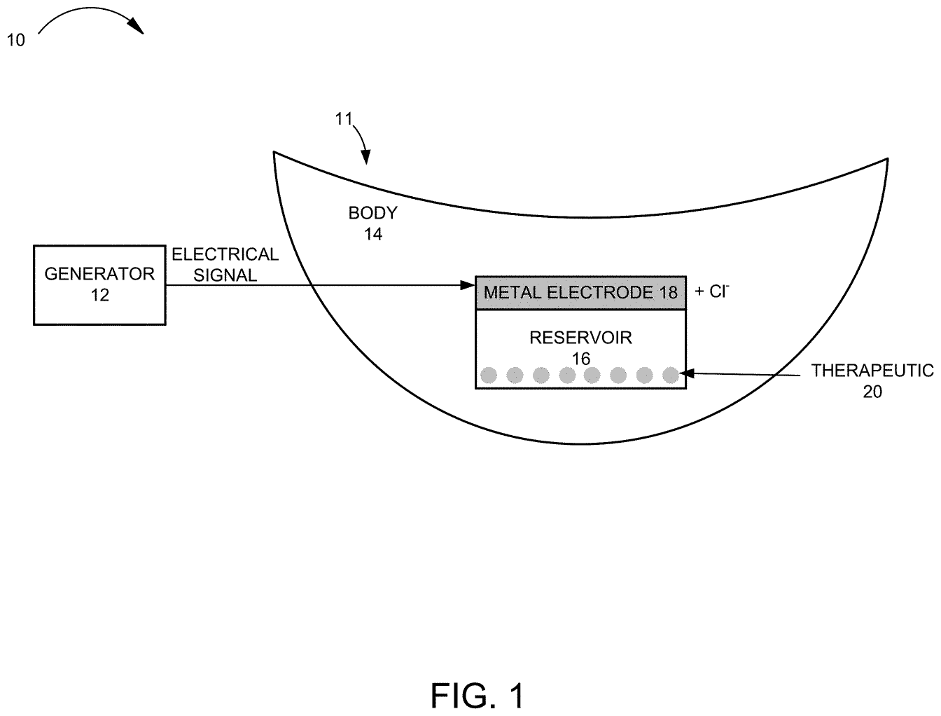

One aspect of the present disclosure includes a system 10 (FIG. 1) that can control the on-demand release of a therapeutic from an ophthalmic device 11 into a patient's eye to treat and/or relieve symptoms of diseases, disorders, or injuries of the eye. The ophthalmic device 11 can store the therapeutic and release the therapeutic using an electrodissolution process. The ophthalmic deice 11 can connect to a generator 12 (e.g., an electrical generator), which can provide an electrical signal that can work as a catalyst that drives the electrodissolution process (the generator 12 may be connected to a controller, not shown, that can, among other things, provide parameters for the electrical signal to be generated). The electrodissolution process can be used to ensure that an amount of therapeutic will be released during a time period, ensuring the on demand release of the therapeutic with a concentration of chloride ions local to the point of electrodissolution for treatment/symptom relief.

The ophthalmic device 11 includes a body 14 encapsulating a reservoir 16 that is covered by a metal electrode 18. The body 14 can be made of a hydrogel matrix formed of a hydrogel-based material and water. The hydrogel-based material can be any cross-linked hydrophilic polymer that does not dissolve in water. Accordingly, the hydrogel-based material can be stiff when dry, but soft and pliable when hydrated. The hydrogel-based material is highly absorbent, and has a naturally-high water content (e.g., 20%-60%), yet maintains a well defined structure. Non-limiting examples of hydrogel-based material monomers are hydroxyethylmethacrylate (HEMA) or derivatives, methacrylic acid (MA) or derivatives, methyl methacrylate (MMA) or derivatives, n-vinyl perrolidone (NVP) or derivatives, poly vinyl alcohol (PVA) or derivatives, polyvinyl pyrrolidone (PVP) or derivatives, and the like. In some instances, the hydrogel-based material can include silicone (as a “silicone-hydrogel”), increasing the oxygen transmissibility and permeability of the hydrogel (among other bulk and surface properties that the presence of silicone improves).

The body 14 can encapsulate at least the reservoir 16 and the metal electrode 18 (other components that could be encapsulated, including reference and counter electrodes, are not shown). The reservoir 16 can be shaped to hold a therapeutic 20 and sized to fit within the volume of the body 14 (for example, the reservoir can have a diameter on the order of tens or hundreds of microns, such as 5 μm, 50 μm, or 500 μm). The reservoir 16 has an interior configured to hold the therapeutic 20. The therapeutic 20 can be a liquid, solid, or gas. The therapeutic 20, for example, can be used for the treatment and/or symptom relief of diseases such as glaucoma and dry eye. The reservoir 16 can be made of photo-patternable polymers such as an epoxy-based negative photoresist material (SU-8), a positive photoresist material (AZ 1500), a cyclic olefin copolymer (COC), a cyclic olefin polymer (COP), or other thermoplastic polymers such as liquid crystal polymer (LCP), Parylene, Polyimide, polypropylene, polycarbonate, Ultem or Nylon. The reservoir 16 includes at least a portion being open, allowing the therapeutic 20 to diffuse out of the reservoir 16 and into the surrounding hydrogel matrix. To prevent the release of the therapeutic 20 from the reservoir 16, the opening can be covered, for example by the metal electrode 18. The metal electrode 18 can include one or more electrochemically active metal. One example of such a metal is gold. The gold can be thin enough to facilitate the electrodissolution, like the non-limiting example of a gold film electrode.

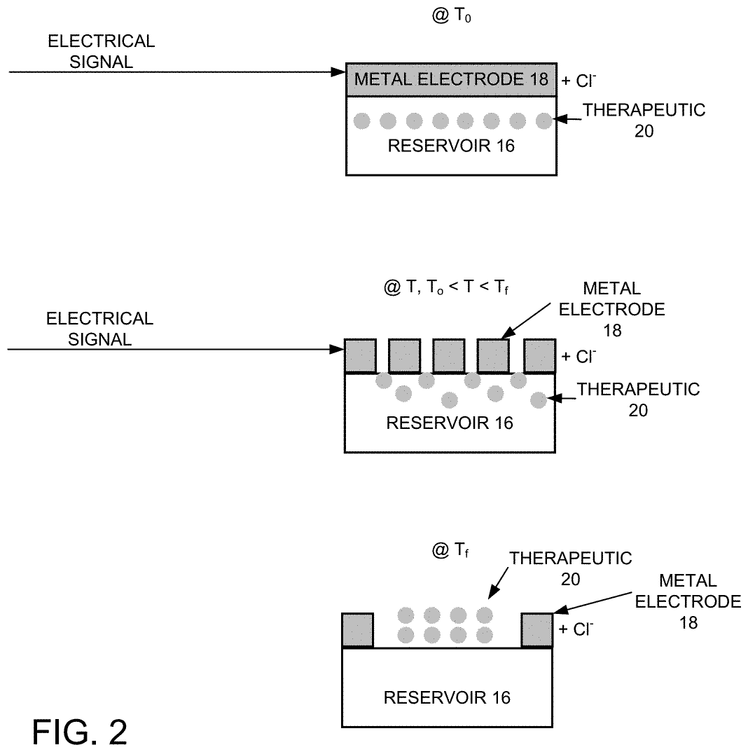

The generator 12 can configure and transmit an electrical signal (which can be a current signal and/or a voltage signal) to at least the metal electrode 18. The generator 12 can transmit the electrical signal over a wired connection, a wireless connection, or a combination of wired and wireless connection. The metal electrode 18 can undergo electrodissolution in response to the application of the electrical signal from the generator 12. The metal electrode 18 can be connected to the generator 12 to receive the electrical signal, which can cause electrodissolution of the metal electrode 18. A non-limiting example of electrodissolution of the metal electrode 18 (shown, for example, in a pictorial representation in FIG. 2) can facilitate the release of the therapeutic 20 from the reservoir 16 and can be enhanced by the presence of chloride ions, the electrode can also dissolve in other ways not shown.

FIG. 2 illustrates how electrodissolution of the metal electrode 18 occurs in the presence of chloride ions to release the therapeutic 20 from the reservoir 16. At time T₀ (start time) the electrical signal starts to be applied to the metal electrode 18 by the generator 12 (not shown in FIG. 2). At time T₀ the reservoir 16 containing the therapeutic 20 is entirely closed off by the metal electrode 18 so that no therapeutic 20 can escape. The electrical signal is applied through time T, which is between times T₀ and T_{f} (final time), where the metal electrode 18 in the presence of chloride ions begins to dissolve in response to the applied electrical signal. At time T the therapeutic 20 can be at least partially still held within the reservoir 16. At time T_{f} the metal electrode 18 is electrodissolved such that the metal electrode no longer covers the opening of the reservoir 16 and the therapeutic 20 can diffuse out from the reservoir 16. When the metal electrode 18 has electrodissolved at time T_{f} the electrical signal is ended. After the electrodissolution (or, in some instances, at any point when the metal electrode has dissolved a sufficient amount), the therapeutic 20 can freely diffuse out of the opening of the reservoir 16 it can then diffuse out of the body 14 of FIG. 1 (not shown in FIG. 2) onto and/or into the eye of the subject wearing the ophthalmic device 11 of FIG. 1 (not shown in FIG. 2).

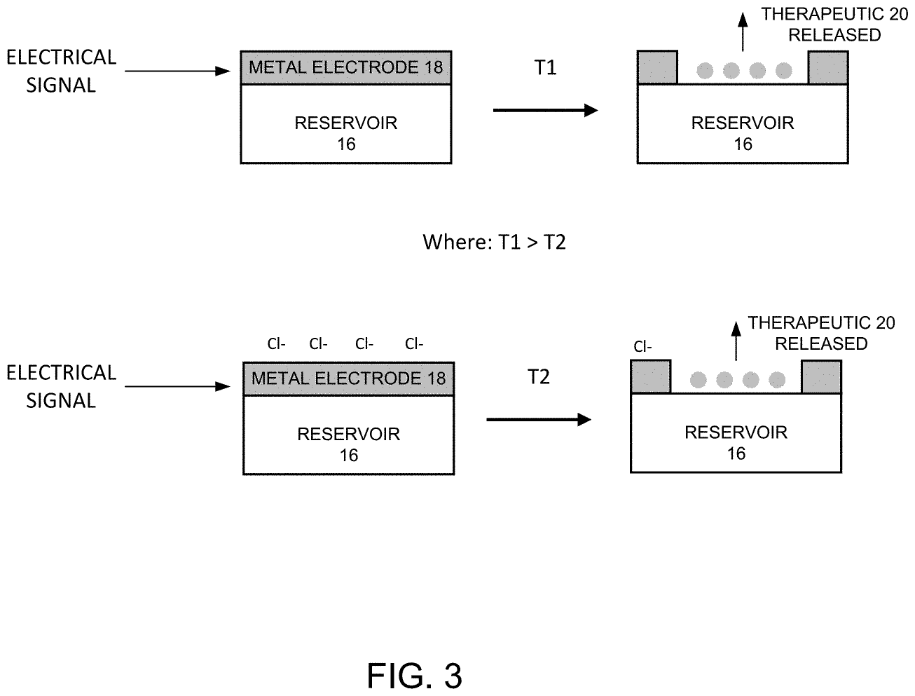

FIG. 3 illustrates the importance of chloride ions for a timely electrodissolution process. The top portion of FIG. 3 shows electrodissolution of the metal electrode 18 without the presence of chloride ions proximal the surface of the metal electrode facing away from the reservoir 16. Without the presence of chloride ions the metal electrode 18 takes time T₁ to dissolve fully in response to the application of the electrical signal and to release the therapeutic 20 from the reservoir 16. The bottom portion of FIG. 3 shows electrodissolution of the metal electrode 18 in the presence of chloride ions proximal the surface of the metal electrode facing away from the reservoir 16. In the presence of chloride ions the metal electrode 18 takes time T₂ to fully dissolve in response to the application of the electrical signal and to release the therapeutic 20 from the reservoir 16. Time T₂ is less than time T₁ because the chloride ions improve the efficiency of the electrodissolution process. The amount of current that can be generated at the metal electrode 18 for an electrical signal of a given applied voltage can be based on the amount of chloride ions locally available to the metal electrode until a maximum current for the system and voltage is reached.

By encasing the metal electrode within a traditional silicone-hydrogel or hydrogel-based material the chloride ions from a saline solution in contact with the ophthalmic device can be impeded from reaching the proximity of the embedded metal electrode. Three techniques of enhancing the chloride ion concentration at the local environment of a metal electrode surrounded by a hydrogel-based material within the ophthalmic device are described. The increased chloride concentration proximal the metal electrode can be provided by the hydrogel-based material of the body 14 having an enhanced permeability to chloride and/or the presence of a boost layer that includes a material with high water solubility and/or a solid salt.

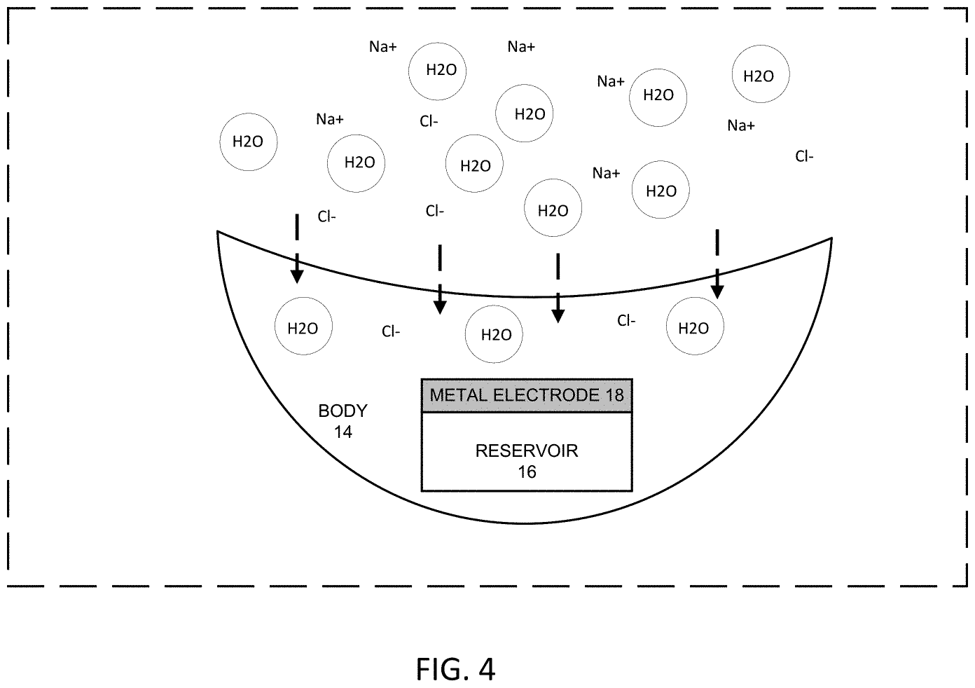

The enhanced concentration of chloride ions can be provided local to the metal electrode 14 when the hydrogel-based material of the body 14 is engineered with an increased permeability to the chloride ions, as shown in FIG. 4. In FIG. 4 the hydrogel-based material of body 14 has an increased permeability to chloride ions. The hydrogel-based body 14 is shown immersed in a saline solution comprising water molecules, sodium ions, and chloride ions. Other solutions containing chloride ions could also be used. At least a portion of the chloride ions and water molecules can permeate through the body 14 towards the metal electrode 18. The chloride permeability of the hydrogel-based material of the body 14 determines the quantity of chloride ions that can reach the metal electrode in a given amount of time. The higher the chloride permeability the more chloride ions that can permeate towards the metal electrode 18 in a given amount of time. For example, for a material to have a high chloride permeability the permeability is at least 400×10⁸ cm²/s, preferably from about 400×10⁸ cm²/s to 700×10⁸ cm²/s.

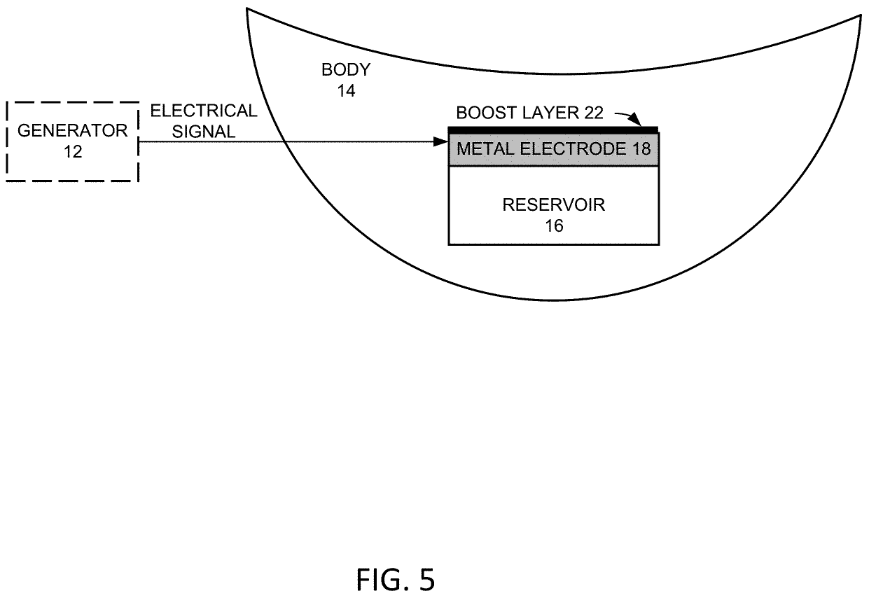

FIG. 5 illustrates another example of how to increase the quantity of chloride ions proximal to the metal electrode 18 via a boost layer 22 (made of one or more materials with a high water solubility and/or a solid salt). The boost layer 22 is located proximal to the metal electrode 18, on a side of the metal electrode facing away from the reservoir 16. The boost layer 22 is also fully encapsulated within the body 14. The boost layer 22 supplies chloride ions proximal to the metal electrode 18 to enhance the electrodissolution process. The chloride ions can be supplied from at least one of: the composition of the boost layer 22, chloride ions stored underneath the boost layer and released, or chloride ions collected from the local environment of the ophthalmic device 11 (e.g., saline solution, tears, etc.). Specifically, the boost layer 22 dissolves to form a local enhanced chloride environment that leads to an increase in a speed of the electrodissolution.

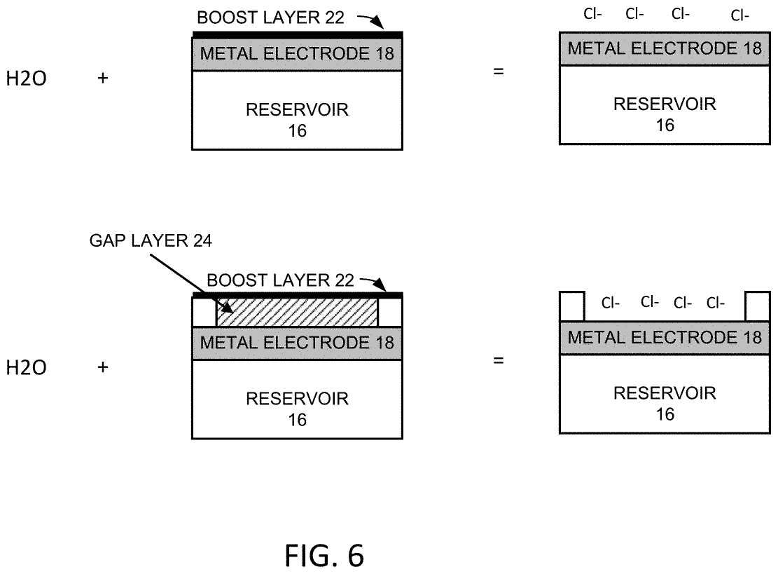

FIG. 6 illustrates two examples of how the boost layer 22 can be configured proximal the metal electrode 18 and how the boost layer reacts in the presence of water molecules. The top portion of FIG. 6 shows the boost layer 22 directly on top of/above the metal electrode 18 (e.g. on a side of the metal electrode not facing the reservoir 16). An example of a boost layer 22 directly on top of/above the metal electrode 18 is a solid salt comprising chloride ions. Examples of a solid salt include, but are not limited to, sodium chloride (NaCl) or potassium chloride (KCl).

The bottom portion of FIG. 6 shows the boost layer 22 separated from the metal electrode 18 by a gap layer 24. In this case the boost layer 22 includes a material with a high water solubility configured to create the gap layer 24 that enables storage of a chloride containing material. The chloride containing material can be a solid, gas, or aqueous solution. The boost layer 22 and accompanying gap layer 24 are on a side of the metal electrode 18 not facing the reservoir 16. In one example, the high water soluble material of the boost layer 22 can be a material with a water solubility greater than or equal to a lower limit. One example is greater than a lower limit of 1 g/L. High water soluble materials include, but are not limited to, Polyvinyl alcohol (PVA), Polyvinylpyrrolidone (PVP), Poly ethylene glycol (PEG), or Polyacrylic acid (PAA). In another example, in the case of an ophthalmic device that is stored in an aqueous solution for several weeks or months, or more, before use, then the lower limit can be below 1 g/L (e.g., depending on the expected length of storage before use) so the boost layer 22 does not dissolve prior to use of the ophthalmic device. The boost layer 22 can be held off of the metal electrode 18 by a similar material used to construct the reservoir 16. When water is added to either boost layer 22 example shown in FIG. 6, the boost layer 22 will at least partially dissolve leaving behind an enhanced local chloride environment proximal to the metal electrode 18. When the electrical signal is applied to the metal electrode 18 by the generator 12, the metal electrode will dissolve quicker due to the increase of chloride ions proximal to the electrodissolution reaction. The boost layer configurations can be utilized together. By way of non-limiting example, the boost layers can be layered such that the solid salt layer is inside the gap layer formed by the high water soluble boost layer.

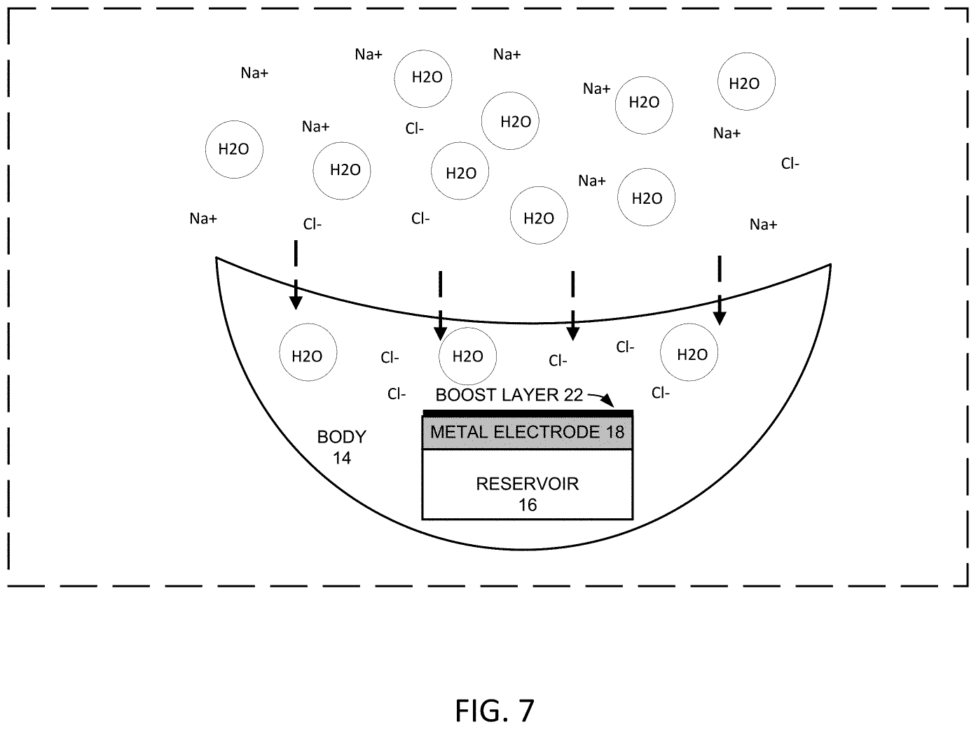

FIG. 7 shows an example of the ophthalmic device 10 which includes a body 14 made of the hydrogel-based material that fully encapsulates the boost layer 22, the metal electrode 18, and the reservoir 16. At least water molecules and chloride ions can diffuse and permeate across the body 14 towards the boost layer 22 when the ophthalmic device 10 is at least partially immersed in a saline, or other biocompatible chloride containing solution. The hydrogel-based material of the body 14 can include a silicone-hydrogel or a hydrogel having a high chloride permeability (e.g., a permeability greater than or equal to 400×10⁸ cm²/s, preferably from about 400×10⁸ cm²/s to 700×10⁸ cm²/s, and the boost layer 22 can be a material with a high water solubility and/or a solid salt.

### IV. Methods

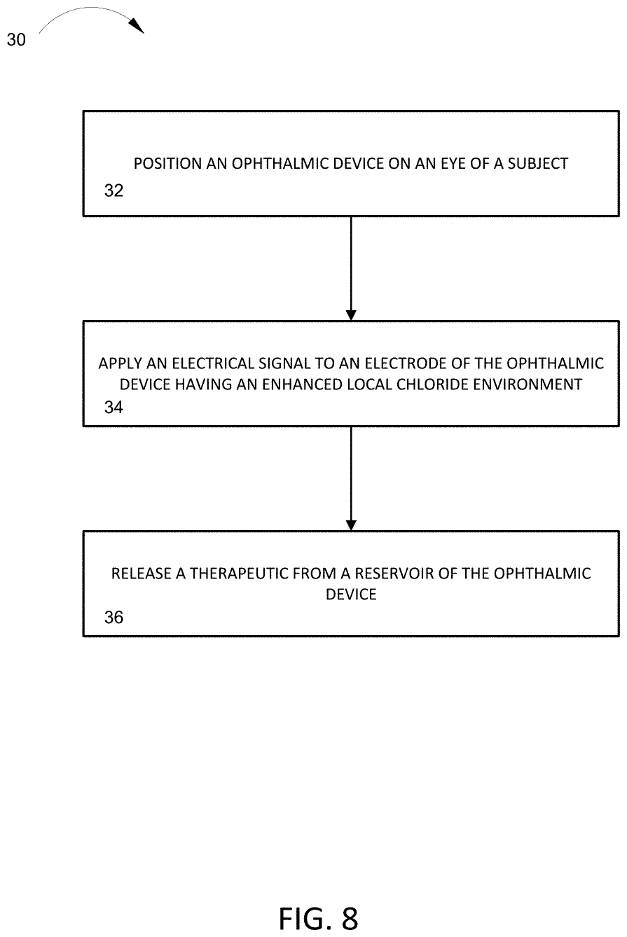

Another aspect of the present disclosure can include methods 30, 40, and 50 (FIGS. 8-10) for controlling the release of a therapeutic from an ophthalmic device by enhancing the quantity of chloride ions in the local environment of a metal electrode within the ophthalmic device. The methods 30, 40, and 50 can be executed using the system 10 and the ophthalmic device 11 described above with respect to FIGS. 1-7.

The methods 30, 40, and 50 are illustrated as process flow diagrams with flowchart illustrations. For purposes of simplicity, the methods 30, 40, and 50 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 30, 40, and 50.

Referring now to FIG. 8, illustrated is a method 30 for controlling the release of a therapeutic from a smart ophthalmic device by enhancing the quantity of chloride ions in the local environment of a metal electrode within the ophthalmic device. At 32, an ophthalmic device is positioned on an eye of a subject. The ophthalmic device can be the ophthalmic device 11 of FIG. 1 and includes a reservoir and a metal electrode encapsulated within a body of the ophthalmic device. The reservoir can have an interior configured to hold a therapeutic. The metal electrode can cover an opening of the reservoir and can be electrodissolved to release the therapeutic from the reservoir. The body can include a hydrogel-based material (e.g., hydroxyethylmethacrylate (HEMA) or derivatives, methacrylic acid (MA) or derivatives, methyl methacrylate (MMA) or derivatives, n-vinyl perrolidone (NVP) or derivatives, poly vinyl alcohol (PVA) or derivatives, polyvinyl pyrrolidone (PVP) or derivatives, and the like), which may include silicone, that encapsulates the reservoir and the metal electrode. The hydrogel-based material can be permeable to chloride ions. In one example, the hydrogel-based material can have a high chloride permeability (e.g., a permeability greater than or equal to 400×10⁸ cm²/s, preferably from about 400×10⁸ cm²/s to 700×10⁸ cm²/s. At 34, an electrical signal can be applied to the electrode so that the electrode undergoes electrodissolution to release the therapeutic and treat an eye disease or injury and/or relieve a symptom thereof, where the electrodissolution is enhanced by chloride ions. For example, the eye disease being treated can be one of glaucoma and dry eye. When applying the electrical signal electrodissolution can begin once a charge transfer limit of the electrode is reached. At 36, the therapeutic can be released from the reservoir of the ophthalmic device when the electrodissolution that is enhanced by the presence of chloride ions dissolves at least a portion of the metal electrode.

The ophthalmic device can further include a boost layer proximal to the metal electrode to supply the chloride ions to enhance the electrodissolution. The boost layer can be deposited proximal the electrode by at least one of photolithography, jet-dispensing, inking, pattern transfer, screen printing, dispensing, pick and place, and deposition by evaporation. The boost layer can be a solid salt comprising chloride ions (e.g., NaCl or KCl) that can be positioned directly above the metal electrode. When the solid salt dissolves chloride ions will be created from the reaction. The boost layer can also be a material with a high-water solubility, where a high water solubility is a water solubility greater than or equal to a lower limit, for example, a lower limit of 1 g/L. Examples of high-water soluble materials can include, but are not limited to PVA (Polyvinyl alcohol), PVP (Polyvinylpyrrolidone), PEG (Poly ethylene glycol), or PAA (Polyacrylic acid). The boost layer of a high-water soluble material can be positioned above the metal electrode to create a gap layer 24 that enables storage of a chloride containing material between the boost layer and the metal electrode. The chloride containing material can be a solid, a gas, or an aqueous solution. When the high-water soluble material dissolves the chloride ions in the chloride containing material will be available for reaction during electrodissolution.

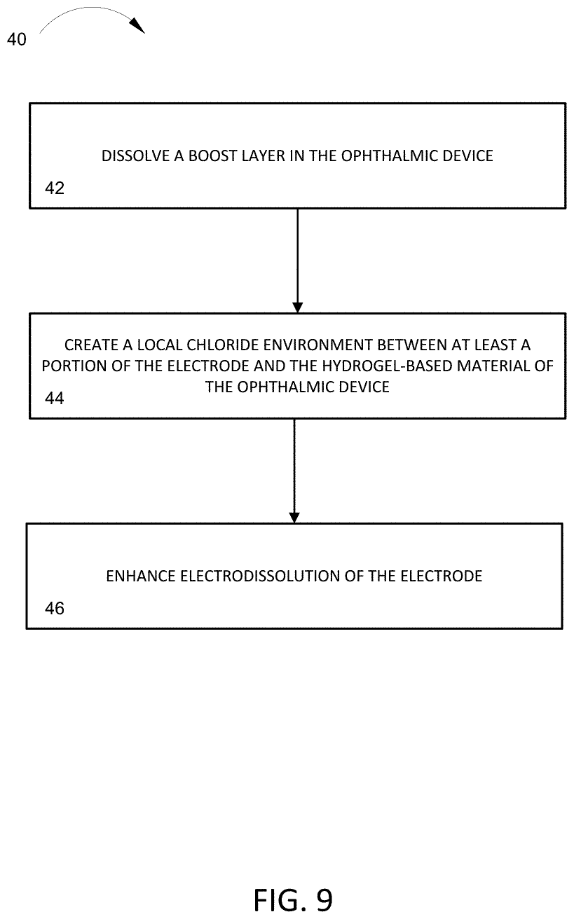

Referring now to FIG. 9, is a method 40 for controlling the release of the therapeutic by enhancing the electrodissolution of the electrode. At 42, the boost layer, which can include at least one of the solid salt or the high-water soluble material, in the ophthalmic device can be dissolved. The boost layer can be dissolved with water (e.g., tears) from the eye of the subject wearing the ophthalmic device diffusing through the hydrogel-based material of the body. At 44, a local chloride environment can be created between at least a portion of the electrode and the hydrogel-based material (e.g., at the electrode/electrolyte interface). The local chloride environment can be created by dissolving the boost layer in the presence of water, which can diffuse through the body of the ophthalmic device from, for example tears on the eye of a subject. The local chloride environment can have a concentration of chloride ions greater than or equal to a concentration of chloride ions in saline from the eye of the subject. At 46, the local chloride environment can enhance the electrodissolution of the electrode and make the electrodissolution quicker and/or more efficient than if less chloride ions were present. When the boost layer has dissolved, an amount of charge transferred from the applied current to the electrode at a time can increase. Dissolving the boost layer can increase an amount of chloride ions proximal the electrode, wherein the amount of chloride ions is proportional to the amount of current generated at a time.

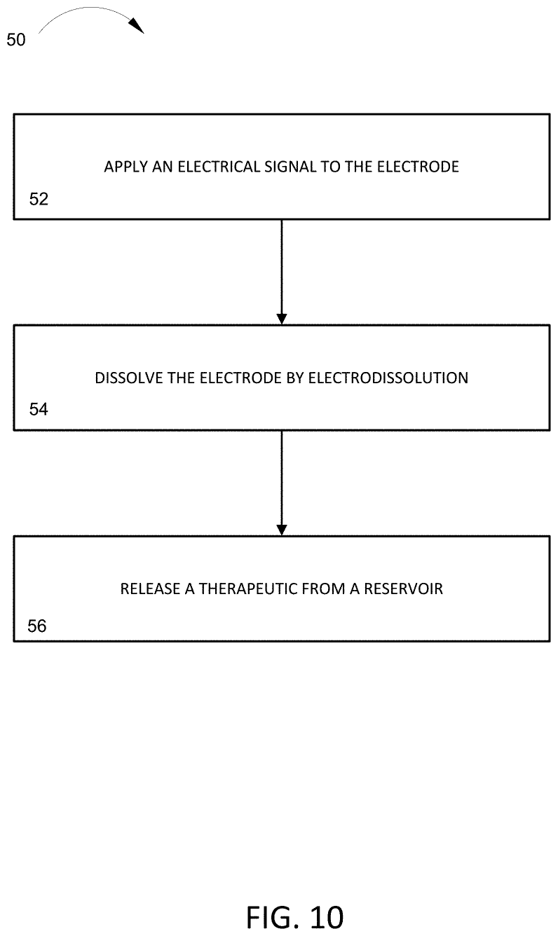

Referring now to FIG. 10, is a method 50 for treating a disease or injury of the eye, or relieving a symptom thereof. This method can be triggered manually by a subject or medical professional, or automatically based on health data from sensors on and/or in the subject. At 52, an electrical signal can be applied to the metal electrode within the ophthalmic device to begin electrodissolution. The electrodissolution can be enhanced by chloride ions proximal the metal electrode that were supplied by at least one of the boost layer, the gap layer, or the saline (e.g., from tears). The electrical signal can be applied from a generator, which can be attached to a controller (e.g., computer, smartphone, a smart wearable accessory, etc.) that can communicate a start time and parameters of the electrical signal being applied. At 54, the metal electrode can be at least partially dissolved by the electrodissolution. At 56, the therapeutic stored in the reservoir can be released from the reservoir. The released therapeutic can diffuse across the hydrogel-material of the body into the eye of the subject to treat the eye disease or injury. The therapeutic can reach an intended treatment target in the eye within a given time (e.g., 30 seconds, one minute, five minutes, ten minutes, etc.) after application of the electrical signal.

From the above description, those skilled in the art will perceive improvements, changes and modifications. Such improvements, changes and modifications are within the skill of one in the art and are intended to be covered by the appended claims.

The following is claimed:

1. An ophthalmic device comprising:
a reservoir having an interior configured to hold a therapeutic;
a metal electrode configured to cover an opening of the reservoir and to receive an electrical signal that electrodissolves the metal electrode to release the therapeutic from the reservoir; and
a body comprising a hydrogel-based material configured to encapsulate the reservoir and the metal electrode,
wherein electrodissolution is enhanced by chloride ions.

2. The ophthalmic device of claim 1, wherein the hydrogel-based material is permeable to the chloride ions.

3. The ophthalmic device of claim 1, further comprising a boost layer located proximal to the metal electrode configured to supply the chloride ions to enhance the electrodissolution.

4. The ophthalmic device of claim 3, wherein the boost layer comprises a material with a high water solubility configured to create a gap layer with the metal electrode that enables storage of a chloride containing material.

5. The ophthalmic device of claim 3, wherein the boost layer comprises a solid salt comprising the chloride ions.

6. The ophthalmic device of claim 3, wherein the hydrogel-based material comprises a hydrogel having a high chloride permeability, and
wherein the boost layer comprises a material with a high water solubility and/or a solid salt.

7. The ophthalmic device of claim 3, wherein the boost layer is configured to dissolve to form a local enhanced chloride environment that leads to an increase in a speed of the electrodissolution.

8. A method comprising:
positioning an ophthalmic device on an eye of a subject, wherein the ophthalmic device comprises:
a reservoir having an interior configured to hold a therapeutic;
a metal electrode configured to cover an opening of the reservoir and to electrodissolve to release the therapeutic from the reservoir; and
a body comprising a hydrogel-based material configured to encapsulate the reservoir and the metal electrode; and
applying an electrical signal to the electrode so that the electrode undergoes electrodissolution to release the therapeutic,
wherein the electrodissolution is enhanced by chloride ions.

9. The method of claim 8, wherein the hydrogel-based material is permeable to chloride ions.

10. The method of claim 8, wherein the ophthalmic device further comprises a boost layer proximal to the metal electrode to supply the chloride ions to enhance the electrodissolution.

11. The method of claim 10, further comprising dissolving the boost layer with water from the eye of the subject diffused through the hydrogel-based material.

12. The method of claim 11, further comprising creating a local chloride environment between at least a portion of the metal electrode and the hydrogel-based material, wherein the local chloride environment enhances the electrodissolution of the metal electrode.

13. The method of claim 12, wherein creating the local chloride environment further comprises creating a concentration of chloride greater than or equal to a concentration of chloride in saline from the eye of the subject.

14. The method of claim 10, wherein the boost layer increases the speed of the electrodissolution by:
dissolving in the presence of water; and
when dissolved, increasing an amount of charge transferred from the applied current to the metal electrode at a time.

15. The method of claim 8 further comprising:
applying an electrical signal to the metal electrode to begin electrodissolution;
dissolving the metal electrode by electrodissolution; and
releasing the therapeutic from the reservoir to diffuse across the hydrogel body into the eye.

16. The method of claim 8, further comprising:
depositing a boost layer proximal the metal electrode by at least one of photolithography, jet-dispensing, inking, pattern transfer, screen printing, dispensing, pick and place, and deposition by evaporation.

17. The method of claim 8, wherein dissolving the boost layer further comprises:
increasing an amount of chloride ions proximal the metal electrode, wherein the amount of chloride ions is proportional to the amount of current generated at a time.

18. The method of claim 8, wherein the applying electrical signal further comprises starting the electrodissolution once a charge transfer limit is reached.

19. The method of claim 8 further comprising treating an eye disease with the therapeutic.

20. The method of claim 19, wherein the eye disease is one of glaucoma and dry eye.
